(19) United States
(12) Patent Application Publication
(10) Pub. No.: US 2021/0236793 A1
(43) Pub. Date: Aug. 5, 2021

(54) MULTI-TUBE ADAPTER

(71) Applicants: YI-CHING LIU, TAIPEI CITY (TW); HSIN-HUA TSAI, TAIPEI CITY (TW); CHIA-FANG CHANG, LOS GATOS, CA (US)

(72) Inventors: YI-CHING LIU, TAIPEI CITY (TW); HSIN-HUA TSAI, TAIPEI CITY (TW); CHIA-FANG CHANG, LOS GATOS, CA (US)

(21) Appl. No.: 16/967,677

(22) PCT Filed: Feb. 5, 2020
(86) PCT No.: PCT/US20/16830

§ 371 Date: Aug. 5, 2020

(51) Int. Cl. A61M 39/10; A61M 39/08; A61M 16/04

(52) U.S. Cl. CPC A61M 39/105; A61M 39/08; A61M 2039/0009; A61M 16/0415; A61M 16/0411

(57) ABSTRACT

The invention discloses a multi-tube body adapter which is connected to a plurality of outer tubes for application to a digestive system and a respiratory system of a human body. The multi-tube body adapter includes a housing to form an accommodating space of the multi-chamber. The plurality of chambers is not in communication with each other. One end of the plurality of chambers respectively provides a connection port and the other end of the plurality of chambers is commonly connected to an extension having a plurality of openings. By connecting with the chamber, the connection port and the opening, a supply path for separately providing the supply path of the respiratory system and the digestive system can be formed.

### FIELD OF THE INVENTION

The present invention relates to medical devices, particularly, to a multi-tube adapter used for application to the digestive system and/or respiratory system of a human body.

### BACKGROUND OF THE INVENTION

Patients with eating or swallowing difficulties generally need a catheter inserted through the nasal cavity, throat and esophagus into the stomach to facilitate input of liquid food or reversely output of the stomach contents for the purpose of relieving stress or medical diagnosis.

Patients with productive cough (i.e. cough with sputum) difficulties generally need a catheter entered through the nasal or oral cavity into the throat and the upper trachea to discharge the sputum by means of negative pressure suction, in order to keep the respiratory tract open.

Patients with hypoxemia need a catheter to deliver high concentration oxygen to the nasal cavity to improve respiratory performance and the oxygen concentration in blood.

Patients with the above needs simultaneously require multiple catheter placements to maintain life supporting functions such as eating, expectoration and breathing.

In view of the above, the present invention provides a multi-tube adapter to provide a new way of caretaking capable of overcoming the aforementioned shortcomings of the prior art.

### SUMMARY OF THE INVENTION

The first objective of the present invention is to provide a multi-tube adapter connected to one or a plurality of outer tubes to establish multiple supply paths, thereby achieving the purpose of supporting human internal organs or discharging secretions (e.g. sputum, saliva, etc.) from inside the human body.

The second objective of the present invention is to provide said multi-tube adapter capable of being independently used in the digestive system or the respiratory system or in both systems simultaneously.

The third objective of the present invention is to provide said multi-tube adapter having a multi-chamber housing where the chambers are not connected with each other, in order to achieve the purpose of preventing different substances (e.g. liquid, medicinal powder, gas, etc.) among the chambers from interfering with one another.

The fourth objective of the present invention is to provide said multi-tube adapter having a plurality of external ends (i.e. multiple connection ports) for respectively connecting to the outer tubes and a single internal end (i.e. the extension as mentioned in the present invention).

The fifth objective of the present invention is to provide said multi-tube adapter wherein the external ends and the internal end are capable of exchanging substances unidirectionally or bidirectionally to achieve the purpose of a single multifunctional device.

The sixth objective of the present invention is to provide said multi-tube adapter wherein the purpose of avoiding misplacement of the outer tubes may be achieved by means of an anti-tube misplacement (or foolproof) mechanism.

The seventh objective of the present invention is to provide said multi-tube adapter wherein adaptation to various functionalities may be achieved by means of adjusting the dimensions of the chambers.

The eighth objective of the present invention is to provide said multi-tube adapter having a closing component to close the supply path to protect the unused supply paths.

The ninth objective of the present invention is to provide said multi-tube adapter wherein the disposition thereof on a patient's nose, face, ears or head may be facilitated by means of a fastener fixing the outer tube.

The tenth objective of the present invention is to provide said multi-tube adapter in combination with electronic components (e.g. control module, sensor, etc.) to achieve the purpose of monitoring, calculating and recording.

The eleventh objective of the present invention is to provide said multi-tube adapter having an indicator disposed at the opening of the connection port for the purpose of indicating the supply path.

The twelfth objective of the present invention is to provide said multi-tube adapter having an extension with a plurality of openings (i.e. providing multiple openings within the caliber of a single tube body) where the length of the extension may be flexibly determined so as to be nearby or inside an organ.

The thirteenth objective of the present invention is to provide said multi-tube adapter wherein the extension may be alternatively connected to an inner tube through which the extension may be disposed nearby or inside an organ.

The fourteenth objective of the present invention is to provide said multi-tube adapter wherein the opening of the extension may be disposed nearby an organ in order to achieve the purpose of directly supplying or extracting (or discharging) substances from the organ or conducting drainage.

The fifteenth objective of the present invention is to provide said multi-tube adapter having a path enabling insertion of an external diagnostic device to achieve the purpose of diagnosing and observing an organ.

The sixteenth objective of the present invention is to provide said multi-tube adapter wherein the outer tube and/or inner tube may be respectively replaced to adapt to various needs as well as achieving efficacies such as cleansing, reducing costs, etc.

In order to achieve the aforesaid objectives among others, the present invention provides a multi-tube adapter which is connected to a plurality of outer tubes for application to a digestive system and a respiratory system of a human body. The multi-tube adapter includes a housing. The housing forms an accommodating space including a first chamber and a second chamber. The first chamber and the second chamber are not connected with each other. Furthermore, the first chamber has a first connection port while the second chamber has a second connection port, and the first chamber and the second chamber have an extension with a first opening and a second opening. The first opening is connected to the first chamber while the second opening is connected to the second chamber. Therein, the first connection port is connected to the first chamber and the first opening, enabling the first connection port to further establish a first supply path with the respiratory system via the first chamber and the first opening; the second connection port is connected to the second chamber and the second opening, enabling the second connection port to further establish a second supply path via the second chamber and the second opening.

Compared to prior art, the multi-tube adapter disclosed in the present invention may be connected to a plurality of outer tubes which provide various functions (e.g. feeding food, supplying oxygen, drawing sputum, injecting medicine) by means of connecting to an external equipment. As the multi-tube adapter disclosed in the present invention provides a plurality of independent chambers, the chambers are capable of executing designated functions independently without interference. Moreover, an extension which is a single tube body connected to multiple chambers is provided in the present invention, and the single tube body provides multiple openings disposed respectively nearby or inside an organ (e.g. stomach, lung, etc.)

In another embodiment, a plurality of foolproof mechanisms such as structure differentiation (e.g. tube diameter, tube shape, etc.), identity differentiation (e.g. color, text, pattern, etc.) are provided to avoid misplacement of the tubes which may cause damage to a patient's organ or endanger his/her life.

In yet another embodiment, by flexibly adjusting the dimensions of the openings of the chamber and/or extension, different needs may be addressed to, for example, the dimensions of an opening required by gas is smaller than that required by liquid.

In another embodiment, a sensor or a control module is provided, to intellectualize the multi-tube adapter for use in monitoring, recording, displaying the status related to each chamber of the multi-tube adapter.

### BRIEF DESCRIPTION OF THE DRAWINGS

FIG. 1 is a sectional structure diagram of a first embodiment of the multi-tube adapter of the present invention.

FIG. 2 is a schematic diagram of the first embodiment of the multi-tube adapter of the present invention being connected to an outer tube.

FIG. 3 is a structure diagram of a second embodiment of the multi-tube adapter of the present invention.

FIG. 4 is a structure diagram of a third embodiment of the multi-tube adapter of the present invention.

FIG. 5 is a structure diagram of a fourth embodiment of the multi-tube adapter of the present invention.

FIG. 6 is a structure diagram of a fifth embodiment of the multi-tube adapter of the present invention.

FIG. 7 is a structure diagram of a sixth embodiment of the multi-tube adapter of the present invention.

### DETAILED DESCRIPTION OF THE PREFERRED EMBODIMENTS

In order to fully understand the objectives, features, and functions of the present invention, the present invention is described in detail as follows by the following specific embodiments along with the accompanying figures.

In the present invention, “a” or “one” is used to describe the units, elements, and components described herein. This is done for the convenience of description only and provides a general meaning to the scope of the present invention. Therefore, unless stated otherwise, this description should be understood to include one, at least one, and more than one.

The context of the present invention are as follows. The terms “comprising”, “including”, “having” or “containing” are intended to encompass non-exclusive inclusions. For example, a component, structure, article, or device that comprises a plurality of elements is not limited to the elements listed herein but may include those not specifically listed but which are typically inherent in the element, structure, article, or device. Other requirements. In addition, the term “or” means an inclusive “or” unless it is specifically stated otherwise, rather than an exclusive “or”.

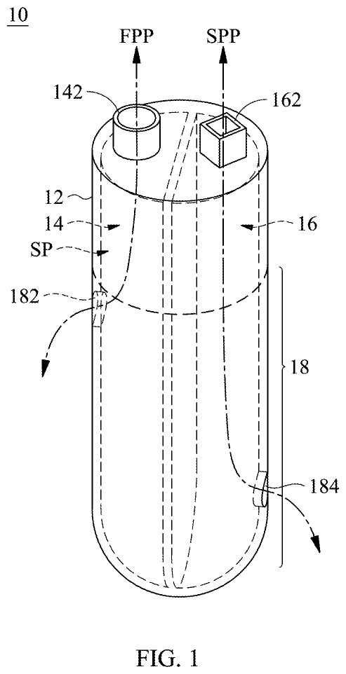

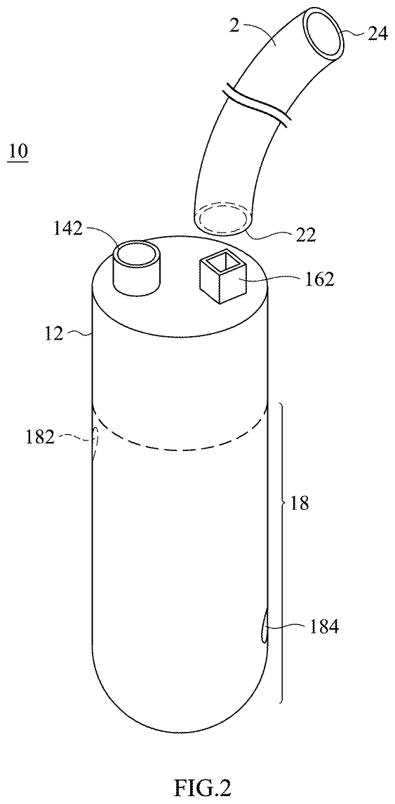

Please refer to FIG. 1, where it is a structure diagram of a first embodiment of a multi-tube adapter of the present invention. In FIG. 1, the multi-tube adapter 10 may be connected to one or a plurality of outer tubes 2 (with reference to FIG. 2) for application to the human digestive system (e.g. esophagus, stomach, intestine, etc.) and respiratory system (e.g. throat, trachea, bronchus, lungs, etc.). An end 22 of the outer tube 2 is connected to the multi-tube adapter 10 while the other end 24 may be selectively connected to another equipment such as a gas cylinder, an external breathing equipment, a negative pressure equipment or a spray equipment, etc. Notably, the outer tube 2 may be freely assembled and disassembled, or be omitted. For example, if the multi-tube adapter 10 needs to obtain oxygen from a gas cylinder, the multi-tube adapter 10 and the gas cylinder may be connected through the outer tube 2 for supply of oxygen; if the function of the multi-tube adapter 10 is to provide nebulized medicine, the medicine may be directly received without the use of the outer tube 2.

Back to FIG. 1, the multi-tube adapter 10 includes a housing 12.

The housing 12 forms an accommodating space SP which includes a first chamber 14 and a second chamber 16. Herein, the shape of the housing 12 is tubular for illustration purpose, nevertheless, the housing 12 may be of any shape, size, material, etc. without any constraint in the present invention. The number of chambers is two for illustration purpose, whereas in other embodiments, the number of chambers may be increased to three, four or more. Notably, for illustration, the first chamber 14 is designated to supply gas (or powder) while the second chamber 16 is designated to supply liquid.

Furthermore, the first chamber 14 and the second chamber 16 are not connected to each other, herein the formation method of the first chamber 14 and the second chamber 16 is not limited, for example, the formation method may be all-in-one or adding a partition board (not shown) in the accommodating space (SP) to segment the first chamber 14 and the second chamber 16. In one embodiment, the dimensions of the first chamber 14 are smaller than or equal to those of the second chamber 16, or the dimensions of the second chamber 16 are smaller than or equal to those of the first chamber 14.

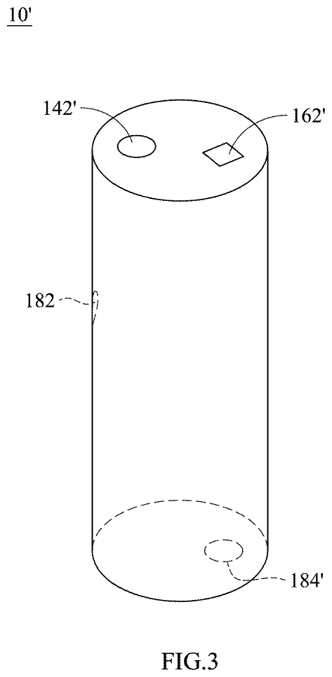

The first chamber 14 has a first connection port 142 while the second chamber 16 has a second connection port 162. Herein, as an example for illustration, the first connection port 142 is of cylindrical shape whereas the second connection port 162 is of rhombic shape, but other embodiments are not limited to the aforesaid shapes. The first connection port 142 and the second connection port 162 may be arranged to have different shapes to achieve the foolproof (or called anti-misplacement) purpose. Moreover, the first connection port 142 and the second connection port 162 have a protruding shape, while in another embodiment, with reference to FIG. 3 of a second embodiment of a multi-tube adapter 10′ wherein the first connection port 142′ and the second connection port 162′ thereof are flat openings.

Back to FIG. 1, the first chamber 14 and the second chamber are connected to an extension 18 having a first opening 182 and a second opening 184, wherein the diameter of the first opening 182 is smaller than or equal to the diameter of the second opening 184; alternatively, the diameter of the second opening 184 is smaller than or equal to the diameter of the first opening 182.

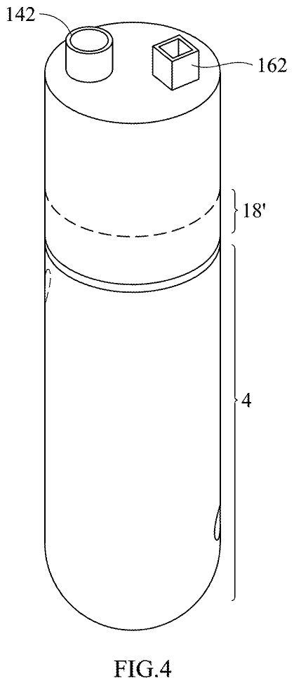

Herein, the first chamber 14 is connected to the first opening 182 and the second chamber 16 is connected to the second opening 184. Furthermore, the first opening 182 may be corresponded to the trachea (not shown) and the second opening 184 may be corresponded to the stomach (not shown). In this embodiment, the extension 18 can directly act as an inner tube, or the extension 18 further connects to another external inner tube 4 (as shown in FIG. 4). Note that the interior of the external inner tube 4 needs to form corresponding space in accordance with the number of openings, that is, the interior of the external inner tube 4 has the extending characteristic of the chamber, and the spaces inside of the external inner tube 4 also have the characteristic of the corresponding chambers in that they are not connected with one another.

Referring back to FIG. 3, the location of the first opening 182 and the second opening 184 are not limited in the present invention; the first opening 182 and the second opening 184 may be provided on the sides of the extension 18 as in FIG. 1, while the second opening 184′ may also be provided at the bottom of the extension 18 as in FIG. 3. In addition, the length and shape of the extension 18 are also not limited. In FIG. 4, as the extension 18′ is able to connect to the external inner tube 4, extension 18′ does not need to be too long. Hence, the length of the extension 18 in FIG. 1 is conspicuously larger than the length of the extension 18′ in FIG. 4.

Back to FIG. 1, the first opening 182 is connected to the first chamber 14 while the second opening 184 is connected to the second chamber 16, wherein the first connection port 142 connects to the first chamber 14 and the first opening 182, enabling the first connection port 142 to further establish a first supply path FPP with, for example, the respiratory system, through the first chamber 14 and the first opening 182; the second connection port 162 is connected to the second chamber 16 and the second opening 184, enabling the second connection port 162 to further establish a second supply path SPP with, for example, the digestive system through the second chamber 16 and the second opening 184.

According to different application scenarios, the first supply path FPP and the second supply path SPP have primarily several configurations, for example: a first configuration in which the first supply path serves to draw secretions such as sputum from the throat through negative pressure to maintain smooth breathing; a second configuration in which the first supply path FPP serves to draw in external medicine powder, medicine spray, oxygen or water vapor spray and supply same to the throat for treatment; a third configuration in which the second supply path acts similarly to a nasogastric tube to provide feeding from outside; a fourth configuration in which the third configuration, if in use, is employed simultaneously with the first configuration or the second configuration. Besides the aforementioned four configurations, other unmentioned configurations are further included.

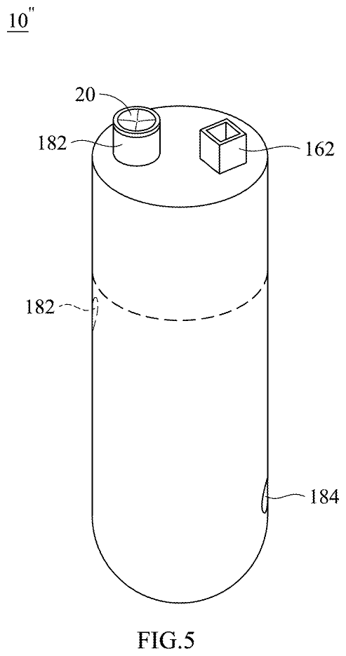

Please refer to FIG. 5, a structure diagram of a fourth embodiment of the multi-tube adapter of the present invention. In FIG. 5, the multi-tube adapter 10″ further includes an open-close component 20 in addition to the housing 12 of the first embodiment.

The open-close component 20 is disposed at the first chamber 14. The open-close component 20 selectively connects to the first connection port 142 and the first opening 182. The first connection port 142 based on the open-close component 20 selectively connects to the first chamber 14 and the first opening 182, enabling the first connection port 142 to further establish the first supply path FPP through the first chamber 14 and the first opening 182.

For example, the open-close component 20 further includes a baffle (not shown) and an elastomer (not shown). The baffle is coupled with the elastomer. One end of the baffle is disposed at the side wall of the first connection port 142 while the other end of the baffle is placed against the other side wall of the first connection port 142, so as to connect the first connection port 142 and the first chamber 14 due to the deformation of the elastomer when an external force is applied to the baffle.

Moreover, the open-close component 20 may have a passive opening structure of which the configuration patterns are: (1) in the natural situation, the open-close component 20 closes the first supply path FPP or the second supply path SPP; and (2) when an outer tube is inserted, for example, the open-close component 20 establishes the first supply path FPP or the second supply path SPP.

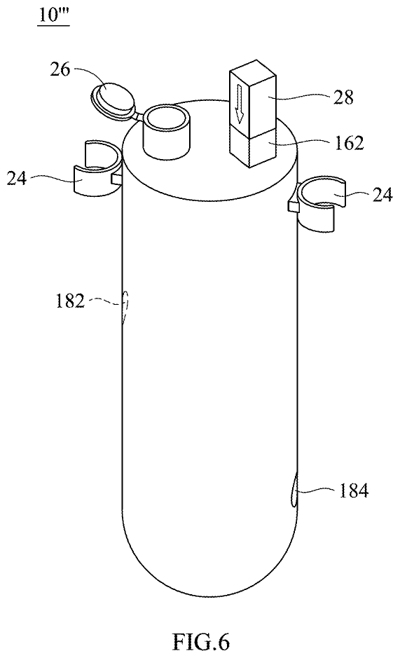

Please refer to FIG. 6, a structure diagram of a fifth embodiment of the multi-tube adapter of the present invention. In FIG. 6, the multi-tube adapter 10″ further includes a fastener 24, a closing component 26 and an indicator 28 in addition to the housing 12 of the first embodiment.

The fastener 24 is disposed on the housing 12 for fixing the housing 12 onto a human body or fixing an outer tube, wherein the fastener 24, for example, may be configured to be a buckle or a c-ring. In the configuration of fixing onto the human body, the multi-tube adapter 10′″ may be fixed to the head via a strap in combination with a buckle; in the fixing the outer tube configuration, the outer tube may be snapped on via the c-ring.

The closing component 26 is couple with the connection port 142 (or the second connection port 162) for selectively closing the first supply path FPP and the second supply path SPP; for example, the close component 26 may be a lid, a cover or a valve, etc., and the lid shall be taken as an example for illustration.

The indicator 28 is disposed at the opening of the first connection port 142 (or the second connection port 162), capable of indicating the supply direction of, for example, the first supply path FPP and the second supply path SPP, and the form thereof may be a sleeve.

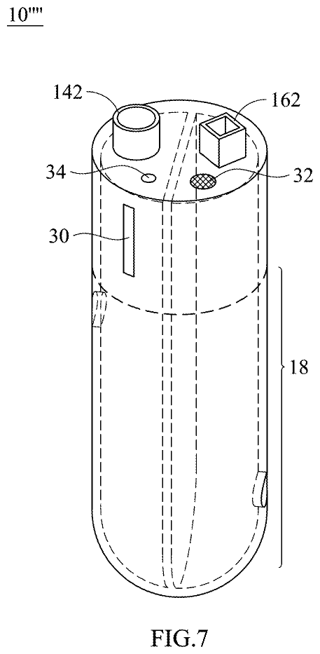

Please refer to FIG. 7, a structure diagram of a sixth embodiment of the multi-tube adapter of the present invention. In FIG. 7, the multi-tube adapter 10″″ further includes a sensor module 30, an output module 32 and an input module 34 in addition to the housing 12 of the first embodiment.

The sensor module 30 is disposed on the housing 12 for detecting the statuses of the first connection port, the second connection port, the first chamber, the second chamber, the first opening, the second opening, the first supply path, the second supply path, wherein the statuses include physical quantities such as gas flow, liquid flow, light source, sound source, temperature, humidity, wind power, oxygen concentration, carbon dioxide concentration, PH value, height, etc. According to the detection of the aforesaid physical quantities, airflow status, feeding counts, feeding time, etc. may be obtained through calculation by an application (APP). The output module is disposed on the housing, the output module emitting light, outputting thermal energy, making sounds, generating micro currents, etc.

The output module 32 is disposed on the housing 12. The output module 32 is capable of emitting light, outputting thermal energy, making sounds, generating micro currents, generating vibrations, and may be, for example, an LED component, an electrothermal coupling component, a speaker, a battery, a motor, etc.

The input module 34 is disposed on the housing 12. The input module 34 captures sound, video and may be a microphone, a photographic lens, etc.

The present invention is disclosed in the abovementioned description by several preferred embodiments, but it is supposed to be comprehended by those who are skilled in the art that the embodiments are used only to illustrate the present invention rather than restrict the scope of the present invention. It should be noted that any equivalent variance or replacement in the embodiments shall be covered by the scope of the present invention. Therefore, what is claimed in the present invention shall be subject to the claims.

1. A multi-tube adapter connected to a plurality of outer tubes for application to human digestive system and respiratory system, the multi-tube adapter being inserted through the nasal cavity, throat and esophagus into the stomach, and including:
a housing forming an accommodating space having a first chamber and a second chamber, the first chamber and the second chamber being not connected to each other, wherein the first chamber has a first connection port and the second chamber has a second connection port, and the first chamber and the second chamber have an extension having a first opening adjacent to the throat and a second opening adjacent to the stomach, the first opening being connected to the first chamber and the second chamber being connected to the second opening;
wherein the first connection port is connected to the first chamber and the first opening, enabling the first connection port to further establish a first supply path with the respiratory system through the first chamber and the first opening, and the second connection port is connected to the second chamber and the second opening, enabling the second connection port to further establish a second supply path with the digestive system through the second chamber and the second opening.

2. The multi-tube adapter according to claim 1, further including an open-close component disposed at the first chamber, the open-close component being selectively to the first connection port and the first opening, the first connection port being connected to the first chamber and the first opening in accordance with the open-close component, enabling the first connection port to further establish the first supply path with the respiratory system through the first chamber and the first opening.

3. The multi-tube adapter according to claim 1, further including a closing component coupled to at least one of the first connection port and the second connection port for selectively closing the first supply path and the second supply path.

4. The multi-tube adapter according to claim 1, further including a fastener disposed at the housing for fixing the housing onto a human body or fixing outer tubes thereon.

5. The multi-tube adapter according to claim 1, further including an indicator disposed at an opening of at least one of the first connection port and the second connection port for indicating the first supply path and the second supply path.

6. The multi-tube adapter according to claim 1, wherein at least of the first connection port and the second connection port has a foolproof mechanism.

7. The multi-tube adapter according to claim 1, further including a sensor module disposed at the accommodating space for detecting the statuses of at least one of the first connection port, the second connection port, the first chamber, the second chamber, the first opening, the second opening, the first supply path, the second supply path, wherein the statuses include at least one of the physical quantities such as gas flow, liquid flow, oxygen concentration, carbon dioxide concentration, PH value, light source, sound source, temperature, humidity, wind power and height.

8. The multi-tube adapter according to claim 1, further including an output module disposed at the housing, the output module being capable of at least one of emitting light, outputting thermal energy, making sounds, generating micro currents, generating vibrations.

9. The multi-tube adapter according to claim 1, further including an input module disposed at the housing, the input module capturing at least one of sound and video.

10. The multi-tube adapter according to claim 1, wherein the location of the second opening of the extension is located on an end portion of the extension, and the first opening of the extension is located on another end portion of the extension or located on a middle portion of the extension.

11. An inner tube connected to a multi-tube adapter, the inner tube being formed as a single tube body inserted through the nasal cavity, throat and esophagus into the stomach and including:
a first chamber and a second chamber separately located inside the inner tube, the first chamber having a first opening adjacent to throat, the second chamber having a second opening in the end portion of the single tube body adjacent to stomach.
